(19) United States
(12) Patent Application Publication
(10) Pub. No.: US 2018/0015195 A1
(43) Pub. Date: Jan. 18, 2018

(54) ODOR MASKING TABLETS

(71) Applicant: Michael N. Brunett, Melbourne, FL (US)

(72) Inventor: Michael N. Brunett, Melbourne, FL (US)

(21) Appl. No.: 15/642,398

(22) Filed: Jul. 6, 2017

Related U.S. Application Data
(60) Provisional application No. 62/362,689, filed on Jul. 15, 2016.

(51) Int. Cl. A61L 9/012

(52) U.S. Cl. CPC A61L 9/012

(57) ABSTRACT

An odor masking tablet composition includes a solid single-use tablet comprising at least one base oil, a reactive agent, a base material, and an emulsifier. The tablet is packaged for individual use and transportation, or multiple tablets are packaged together. The tablets function to rapidly dissolve when deposited into toilet water and produce an active surface barrier for trapping unpleasant odors within the water. The tablets also include a scented material, and a coloring agent for changing the color of the water to which the tablet is placed.

### CROSS-REFERENCE TO RELATED APPLICATIONS

This application claims the benefit of U.S. Application Ser. No. 62/362,689 filed on Jul. 15, 2016, the contents of which are incorporated herein by reference.

### TECHNICAL FIELD

The present invention relates generally to odor reduction, and more particularly to a tablet for masking toilet odors.

### BACKGROUND

The statements in this section merely provide background information related to the present disclosure and may not constitute prior art.

Toilets contain water as an agent to contain and remove human waste such as urine and feces. Unfortunately, the water does little to reduce the unpleasant odors associated with the removal process. Although there are many types of odor masking sprays currently available, each of these require a large canister and function to simply replace the unpleasant odor with a more powerful scent. In either instance, such a lingering scent is immediately recognizable by others and is automatically associated with the waste removal process.

Accordingly, it would be beneficial to provide an odor masking tablet which can be discretely carried by a user and deposited directly into a toilet bowl before use, so as to create a barrier on the surface of the water to contain unpleasant odors.

### SUMMARY OF THE INVENTION

The present invention is directed to an odor masking tablet composition. One embodiment of the present invention can include a solid single-use tablet that is produced through a method of combining at least one base oil, a reactive agent, a base material, and an emulsifier. The tablet can be packaged for individual use and transportation, or can be packaged in bulk packs. The tablet can function to rapidly dissolve when deposited into a body of water, such as a toilet so as to produce an active surface barrier for trapping unpleasant odors within the water.

Another embodiment of the present invention can include a scented material for producing a pleasant aroma, and a coloring agent for changing the color of the water to which the tablet is placed.

This summary is provided merely to introduce certain concepts and not to identify key or essential features of the claimed subject matter.

### DETAILED DESCRIPTION OF THE INVENTION

As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

One embodiment of an odor masking tablet can include a solid, single-use toilet deodorizing agent which can be deposited into a toilet bowl, and can rapidly dissolve e.g., 2-5 seconds, to create an active surface barrier. The active surface barrier consisting of the dissolved tablet can comprise a generally high molecular weight e.g., any molecular weight that is sufficient to allow the composition to float on the surface of a body of water, that can spread across the entirety of the surface of the water. The barrier can function to allow biological waste to pass therethrough, and can immediately thereafter reform the barrier to prevent any unpleasant odors from escaping the surface of the water.

Each tablet can be constructed from a chemical composition, in tablet form, that includes a base oil, a reactive agent, a base material, an emulsifier, a scented material (optional), and a coloring material (optional). In various embodiments, the tablets can be constructed and shipped in packages containing a plurality of different varieties of scent or color, for example, or may be packaged and shipped individually, so as to be carried on one's person or in a purse.

In one embodiment, the base oil can include, comprise or consist of bleached coconut oil, which is an odorless base oil for binding the tablet together to form a solid form. The oil can also assist to disperse and coat the surface of the water with the essential oil used for scenting and masking odors.

In one embodiment, the reactive agent can include, comprise or consist of citric acid, and sodium bicarbonate. The reactive agent functions to dissolve the dry tablet when the same is deposited into the toilet water, by creating a chemical reaction that also releases carbon dioxide bubbles. Moreover, depending on the ratio of reactive agent utilized in the tablet composition, the same can function to adjust the pH level of the toilet water.

In one embodiment, the base material can include, comprise or consist of a combination of sodium bicarbonate, baking soda, cornstarch, and flour. As noted above, when a tablet comes in contact with water, the sodium bicarbonate and citric acid combination react to make carbon dioxide bubbles. This is an acid-base reaction, where baking soda is a weak base and citric acid is a weak acid. The cornstarch and flour act as a “filler” to control the reaction between the baking soda and citric acid. Such a feature enables the tablets to be physically formed and molded when dry, and aids in dispersing the oils over the surface of the water when in use.

In one embodiment, the emulsifier can include, comprise or consist of Microcrystalline cellulose (MCC)—An emulsifier and an anti-clumping agent used to bind the sodium bicarbonate granules together with the citric acid. MCC also functions to bind the oils together, and is a long term stabilizer which helps the powder used in forming the tablets to be easily blended together.

In various embodiments, one or more scented oils can be introduced to the tablet during construction. The scented oils will preferably include some form of natural ingredients and/or essential oils, for example, which can function to mask the unpleasant odors during human evacuation into the toilet.

In various embodiments, any number of different coloring materials can be provided. The coloring materials can be used to provide a distinct color to the tablet(s) and/or the toilet water when the tablet makes contact with the same. The coloring materials can include, but are not limited to various oxide pigment powders, dyes and/or food coloring, for example.

Table 1 illustrates the preferred formulation for the odor masking tablet. Each ingredient is listed along with the preferred relative amounts.

TABLE 1

| INGREDIENT | RATIO |
|---|---|
| Sodium Bicarbonate | 52% |
| Citric Acid | 26% |
| Fractionated Coconut Oil | 11% |
| Essential Oils | 5% |
| Corn Starch | 4% |
| All Purpose Flour | 1% |
| Sucrose | .8% |
| Water | .2% |

By providing a portable single-use tablet, users are able to discretely utilize restroom facilities without the embarrassment of unpleasant odors. Moreover, by actively masking the unpleasant odor at the source, the tablets do not require the use of expensive aromatic dispensers which simply overpower the unpleasant odors.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the terms “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. (canceled)

2. The composition of claim 12, wherein the composition comprises a single tablet that is individually packaged in a wrapping material.

3. The composition of claim 12, wherein the composition comprises a plurality of individual tablets that are packaged in a wrapping material.

4-7. (canceled)

8. The composition of claim 12, further comprising:
a scented material that is configured to produce an aroma when making contact with the water.

9. The composition of claim 8, wherein the scented material comprises at least one essential oil.

10. The composition of claim 12, further comprising:
a coloring material that is configured to change a color of the water.

11. The composition of claim 10, wherein the coloring material is selected from a group comprising:
oxide pigment powder, dye and food coloring.

12. A composition for masking odors, said composition comprising:
a solid single-use tablet that is constructed from
52% by weight sodium bicarbonate;
26% by weight citric acid;
11% by weight fractionated coconut oil;
4% by weight corn starch;
1% by weight all purpose flour;
0.8% by weight sucrose; and
0.2% by weight water,
wherein the composition is configured to rapidly dissolve when deposited into a body of water, and to produce an active surface barrier for trapping an odor within the water.

13. A composition for masking odors, said composition consisting of:
a solid single-use tablet that is constructed from a combination of at least one base oil, a reactive agent, a base material, and an emulsifier,
wherein the composition is configured to rapidly dissolve when deposited into a body of water, and to produce an active surface barrier for trapping an odor within the water.
